(19) United States
(12) Patent Application Publication
(10) Pub. No.: US 2024/0127914 A1
(43) Pub. Date: Apr. 18, 2024

(54) METHODS AND APPARATUS FOR EXTRACTION OF CLINICAL TRIAL DATA FROM AN ELECTRONIC MEDICAL RECORD

(71) Applicant: Abiomed, Inc., Danvers, MA (US)

(72) Inventors: Erik Ian Kroeker, Danvers, MA (US); Jerald Curran, Danvers, MA (US); Samantha Polak, Danvers, MA (US)

(73) Assignee: Abiomed, Inc., Danvers, MA (US)

(21) Appl. No.: 18/488,576

(22) Filed: Oct. 17, 2023

Related U.S. Application Data
(60) Provisional application No. 63/416,964, filed on Oct. 18, 2022.

(51) Int. Cl. G16H 10/60; G16H 10/20

(52) U.S. Cl. CPC G16H 10/60; G16H 10/20

(57) ABSTRACT

Methods and systems for extracting patient information from an electronic medical record (EMR) for use in a clinical trial are described. The method includes receiving an indication that a new clinical trial subject has been identified from a subject identification source, retrieving, from an EMR associated with the new clinical trial subject, patient information, processing the retrieved patient information to de-identify the retrieved patient information thereby generating de-identified patient information, and providing a portion of the de-identified patient information as input to an electronic data capture (EDC) system for the clinical trial.

### CROSS REFERENCE TO RELATED APPLICATIONS

This application claims the benefit under 35 U.S.C. § 119(e) to U.S. Provisional Patent Application No. 63/413,964, filed Oct. 18, 2022, and titled, “METHODS AND APPARATUS FOR EXTRACTION OF CLINICAL TRIAL DATA FROM AN ELECTRONIC MEDICAL RECORD,” the entire contents of which is incorporated herein by reference in its entirety.

### FIELD OF THE INVENTION

This disclosure relates to techniques for extracting clinical trial data from an electronic medical record.

### BACKGROUND

Clinical trials are typically required to satisfy requirements for regulatory approval of new medical devices. However, timely recruitment of study participants is often a substantial barrier to success in clinical trials. Typically, data associated with study participants identified for the clinical trial is manually entered into an electronic data capture (EDC) system during trial enrollment and throughout the study.

### SUMMARY

Disclosed herein are systems and methods for extracting and de-identifying information from an electronic medical record (EMR) or electronic health record (EHR) for use in a clinical trial. The inventors have recognized and appreciated that conventional data entry processes for a clinical trial, in which data is manually entered into an EDC system used for the clinical trial, are laborious, time consuming, and error prone. Monitoring of manual data entry is also laborious, time consuming, and requires frequent interactions with the clinical research site staff to resolve discrepancies. As a result, minimal data from subject EMR/EHRs may be used in clinical trials and when used, significant resources are typically invested to obtain reliable data. Such restrictions tend to limit the frequency with which such data may be collected and the utility of the data collected thereby gating innovation. Some embodiments of the present disclosure may reduce the overall time and cost of executing a clinical trial while increasing the volume (and thereby utility) of data collected from a subject's EMR/EHR.

Some embodiments of the present disclosure may enable a significant reduction in the amount of time required by clinical research collaborators to perform data entry. Some embodiments of the present disclosure may enable a significant reduction in manual transcription errors by reducing manual data entry. Some embodiments of the present disclosure may enable a significant reduction in the amount of data monitoring required for clinical trials (and thereby cost). Some embodiments of the present disclosure may enable a significant increase in the amount of data which can be collected in a clinical trial. In some embodiments of the present disclosure, clinical trial data may be available quicker than through traditional, manual transcription techniques.

In some embodiments, a method of extracting patient information from an electronic medical record (EMR) for use in a clinical trial is provided. The method includes receiving an indication that a new clinical trial subject has been identified from a subject identification source, retrieving, from an EMR associated with the new clinical trial subject, patient information, processing the retrieved patient information to de-identify the retrieved patient information thereby generating de-identified patient information, and providing a portion of the de-identified patient information as input to an electronic data capture (EDC) system for the clinical trial.

In one aspect, the method further includes issuing a request to a subject listing storage component for current subject listing data, and the indication that a new clinical trial subject has been identified is received in response to issuing the request. In another aspect, the subject listing storage component is configured to pull patient information from the subject identification source to generate the current subject listing data. In another aspect, the subject identification source comprises a data source configured to store a plurality of subjects having an implantable medical device. In another aspect, the implantable medical device is an implantable heart pump.

In another aspect, the method further includes displaying a graphical user interface (GUI) on a computing device, wherein the graphical user interface includes a representation of the new clinical trial subject. In another aspect, the method further includes receiving via the GUI, a request to link a medical record patient identifier for the new clinical trial subject to a patient identifier in the EDC system, and linking the medical record patient identifier to the patient identifier in the EDC system in response to receiving the request. In another aspect, the request includes the medical record patient identifier, and linking the medical record patient identifier to the patient identifier in the EDC system includes retrieving from an EMR system, patient information associated with the medical record patient identifier, displaying the retrieved patient information on the GUI, receiving, via the GUI, confirmation that the retrieved patient information is accurate, and linking the medical record patient identifier with the patient identifier in the EDC system in response to receiving confirmation that the retrieved patient information is accurate. In another aspect, linking the medical record patient identifier to the patient identifier in the EDC system further includes generating for the new clinical trial subject, a case record and the patient identifier in the EDC system. In another aspect, the medical record patient identifier comprises a medical resource number (MRN) or a fast healthcare interoperability resources (FHIR) identifier.

In another aspect, the method further includes storing the de-identified patient information in a data store, and providing a portion of the de-identified patient information as input to an EDC system for the clinical trial comprises providing a portion of the de-identified patient information from the data store. In another aspect, storing the de-identified patient information in a data store comprises transferring the de-identified patient information to the data store in accordance with one or more healthcare data transfer standards. In another aspect, the one or more healthcare data transfer standards include HL7 or FHIR. In another aspect, providing a portion of the de-identified patient information as input to an EDC system comprises providing less than all of the de-identified patient information stored in the data store as input to the EDC system.

In another aspect, processing the retrieved patient information to de-identify the retrieved patient information comprises de-identifying the retrieved patient information based, at least in part, on requirements of the clinical trial. In another aspect, processing the retrieved patient information to de-identify the retrieved patient information comprises one or more of redacting information, shifting dates, masking data fields, categorizing data values, hashing data values, or replacing data values with surrogate values. In another aspect, retrieving patient information from an EMR associated with the new clinical trial subject comprises retrieving patient information within a particular time window. In another aspect, retrieving patient information within a particular time window comprises retrieving all patient information from the EMR within the particular time window. In another aspect, the particular time window is selected from the group consisting of a medical facility admission window, a medical device implant procedure window, and a researcher-specified window. In another aspect, the method further includes maintaining an audit trail of all transactions associated with retrieving the patient information, generating the de-identified patient information and providing the portion of the de-identified patient information as input to the EDC system.

In some embodiments, a computerized system for automatically transferring data from an electronic medical record (EMR) system to an electronic data capture (EDC) system for use in a clinical trial is provided. The computerized system includes a subject identification source configured to store an identifier for each of one or more potential clinical trial subjects, subject listing storage configured to pull patient information from the subject identification source to generate a current subject listing data, and a hardware processor. The hardware processor is programmed to request, from the subject listing storage, the current subject listing data, retrieve, from an EMR associated with one or more clinical trial subjects included in the current subject listing data, patient information, wherein the EMR is included in the EMR system, process the retrieved patient information to de-identify the retrieved patient information thereby generating de-identified patient information, and provide a portion of the de-identified patient information as input to the EDC system for the clinical trial.

In one aspect, the system further includes medical records storage, and the hardware processor is further programmed to store the de-identified patient information in the medical records storage, and providing a portion of the de-identified patient information as input to the EDC system comprises providing the portion of the de-identified patient information to the EDC system from the medical records storage. In another aspect, the system further includes a data loader configured to provide the portion of the de-identified patient information from the medical records storage to the EDC system. In another aspect, the data loader is configured to pull the portion of the de-identified patient information from the medical records storage.

In another aspect, the system further includes archive storage, and the hardware processor is further programmed to store the de-identified patient information in the medical records storage. In another aspect, the subject identification source comprises a data source configured to store a plurality of subjects having an implantable medical device. In another aspect, the implantable medical device is an implantable heart pump.

In another aspect, the hardware processor is further programmed to display a graphical user interface (GUI), and the graphical user interface includes a representation of the current subject listing data. In another aspect, the hardware processor is further programmed to receive via the GUI, a request to link a medical record patient identifier for a patient included in the current subject listing data to a patient identifier in the EDC system, and link the medical record patient identifier to the patient identifier in the EDC system in response to receiving the request. In another aspect, the request includes the medical record patient identifier, and linking the medical record patient identifier to the patient identifier in the EDC system includes retrieving from an EMR system, patient information associated with the medical record patient identifier, displaying the retrieved patient information on the GUI, receiving, via the GUI, confirmation that the retrieved patient information is accurate, and linking the medical record patient identifier with the patient identifier in the EDC system in response to receiving confirmation that the retrieved patient information is accurate.

In another aspect, linking the medical record patient identifier to the patient identifier in the EDC system further includes generating for the new clinical trial subject, a case record and the patient identifier in the EDC system. In another aspect, the medical record patient identifier comprises a medical resource number (MRN) or a fast healthcare interoperability resources (FHIR) identifier. In another aspect, processing the retrieved patient information to de-identify the retrieved patient information comprises de-identifying the retrieved patient information based, at least in part, on requirements of the clinical trial. In another aspect, processing the retrieved patient information to de-identify the retrieved patient information comprises one or more of redacting information, shifting dates, masking data fields, categorizing data values, hashing data values, or replacing data values with surrogate values. In another aspect, retrieving patient information from an EMR associated with one or more clinical trial subjects included in the current subject listing data comprises retrieving patient information within a particular time window. In another aspect, retrieving patient information within a particular time window comprises retrieving all patient information from the EMR within the particular time window. In another aspect, the particular time window is selected from the group consisting of a medical facility admission window, a medical device implant procedure window, and a researcher-specified window. In another aspect, the hardware processor is further programmed to maintain an audit trail of all transactions associated with retrieving the patient information, generating the de-identified patient information and providing the portion of the de-identified patient information as input to the EDC system.

### BRIEF DESCRIPTION OF DRAWINGS

FIG. 1 schematically illustrates an architecture of a system for extracting clinical trial data from an electronic medical record, in accordance with some embodiments of the present disclosure.

FIG. 2 is a flowchart of a process for providing de-identified patient information from an electronic medical record (EMR) to an electronic data capture (EDC) system, in accordance with some embodiments of the present disclosure.

FIG. 3 is a flowchart of a process for linking a medical record patient identifier to a patient identifier in an electronic data capture (EDC) system, in accordance with some embodiments of the present disclosure.

FIGS. 4A-4N illustrate portions of a graphical user interface (GUI) that may be used to perform one or more of the techniques described herein.

### DETAILED DESCRIPTION

As described herein, the inventors have recognized and appreciated that conventional data entry processes for clinical trials, in which data is manually entered into an EDC system used for the clinical trial, are laborious, time consuming, and error prone. Monitoring of manual data entry is also laborious, time consuming, and requires frequent interactions with the clinical research site staff to resolve discrepancies. As a result, minimal data from subject EMR/EHRs may be used in clinical trials and when used, significant resources may be invested to obtain reliable data. Such restrictions may limit the frequency with which such data is collected, and the utility of the data collected thereby gating innovation. Accordingly, embodiments of the present disclosure may reduce the overall time and cost of executing a clinical trial while increasing the volume (and thereby utility) of data collected from a subject's EMR/EHR. For example, disclosed herein are systems and methods for extracting and de-identifying information from an electronic medical record (EMR) or electronic health record (EHR) for use in a clinical trial.

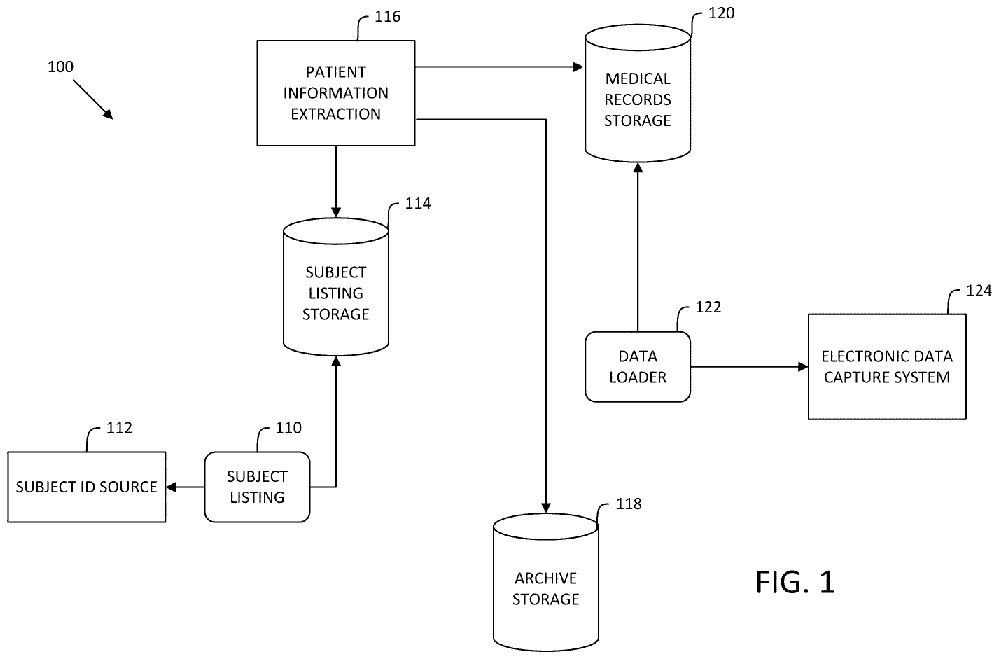

FIG. 1 illustrates an example architecture of a system 100 for extraction of clinical trial data from an EMR/EHR, in accordance with some embodiments of the present disclosure. In some embodiments, a subject listing component 110 may be configured to identify a patient at a clinical trial study site from a subject ID source 112 (e.g., a hospital admission database, an ImpellaConnect® portal available from Abiomed, Inc., Danvers, MA). For instance, the subject listing component 110 may be configured to pull patient information from the subject ID source 112 to generate subject listing data. Subject listing component 110 may be configured to transmit the patient information to subject listing storage 114. For instance, subject listing component 110 may be configured to push subject listing information to subject listing storage 114.

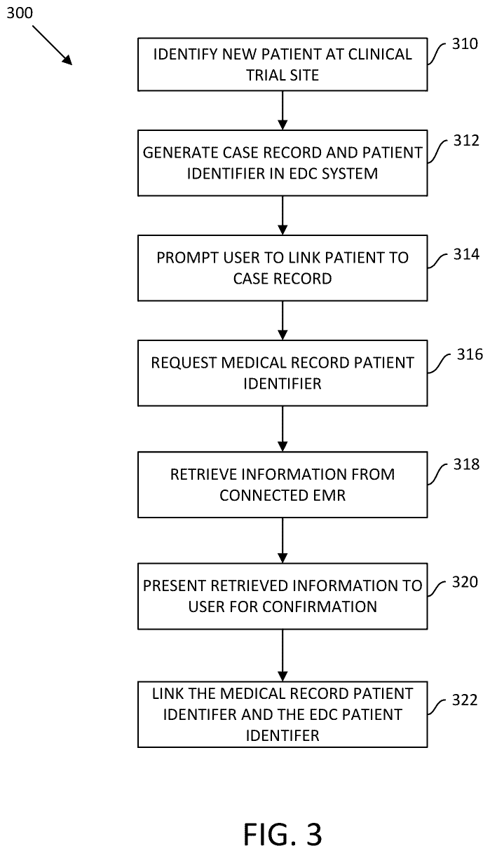

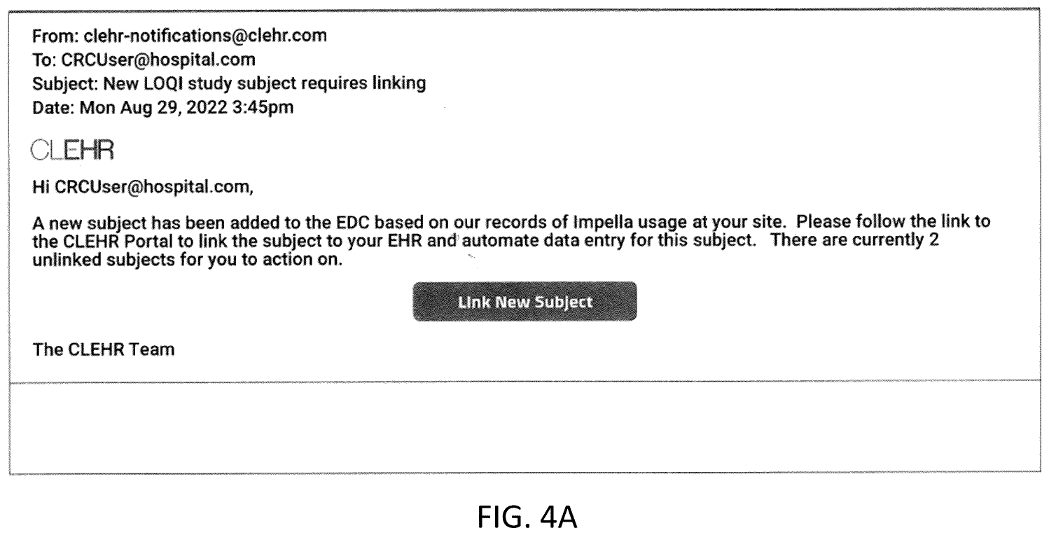

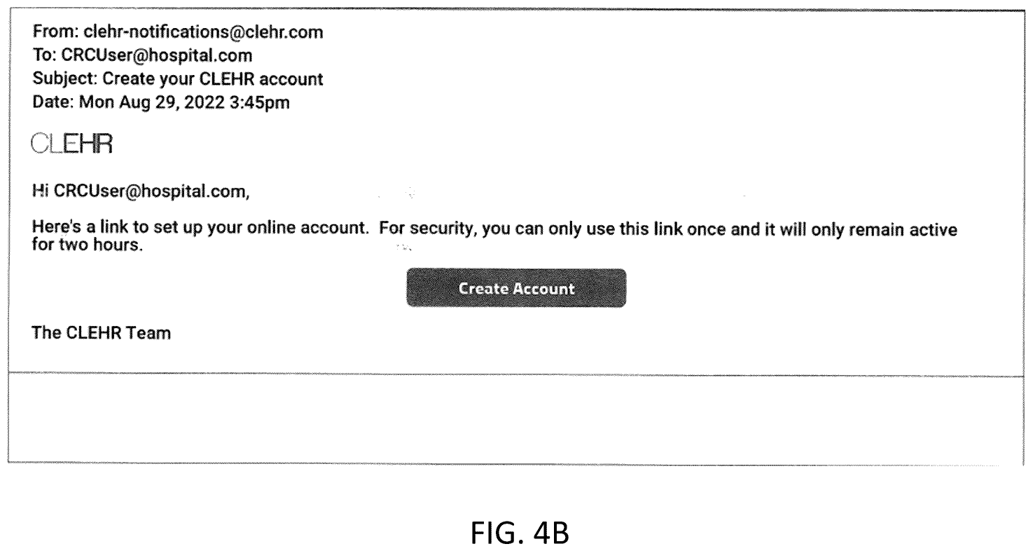

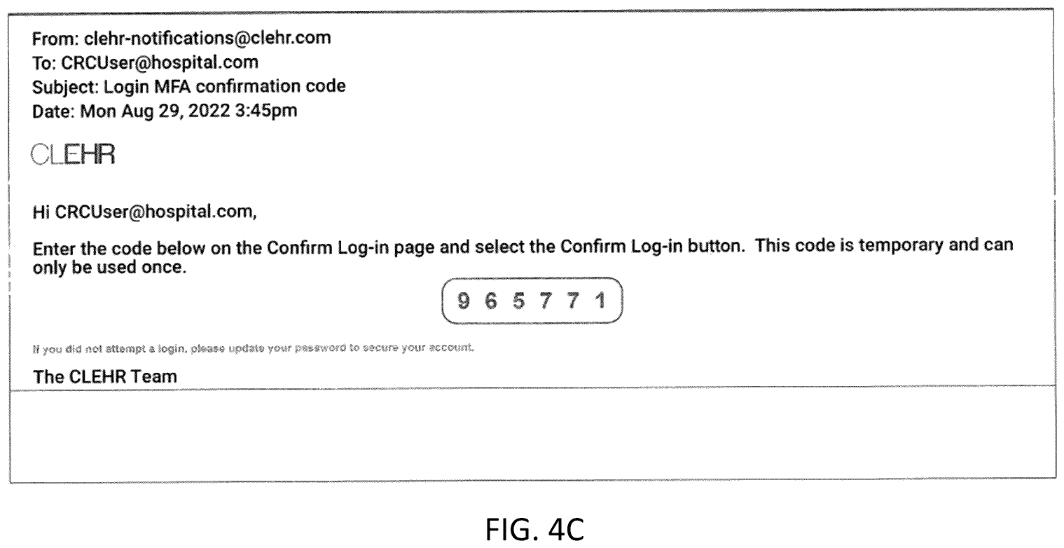

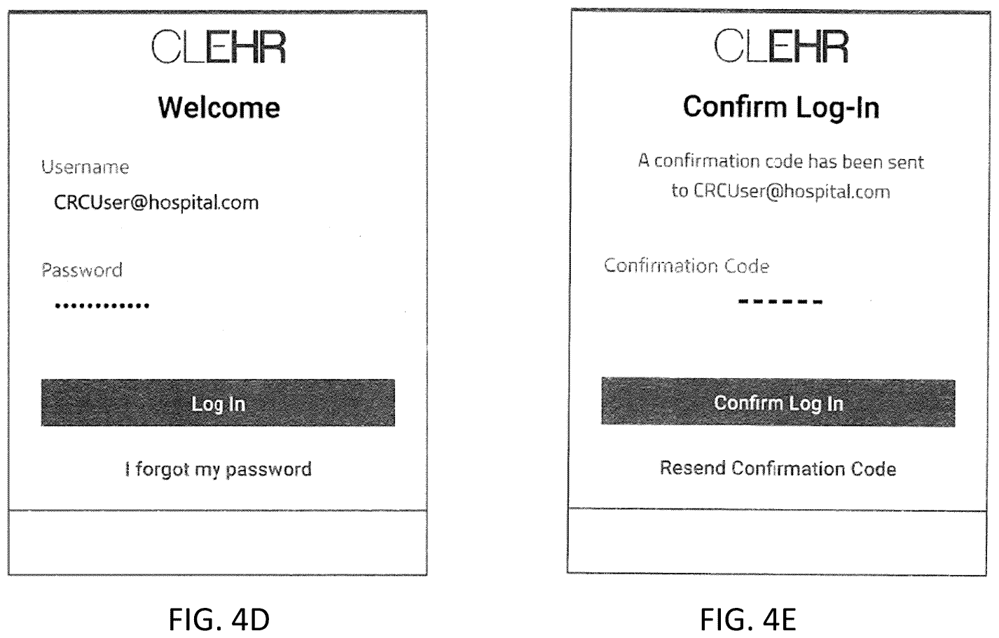

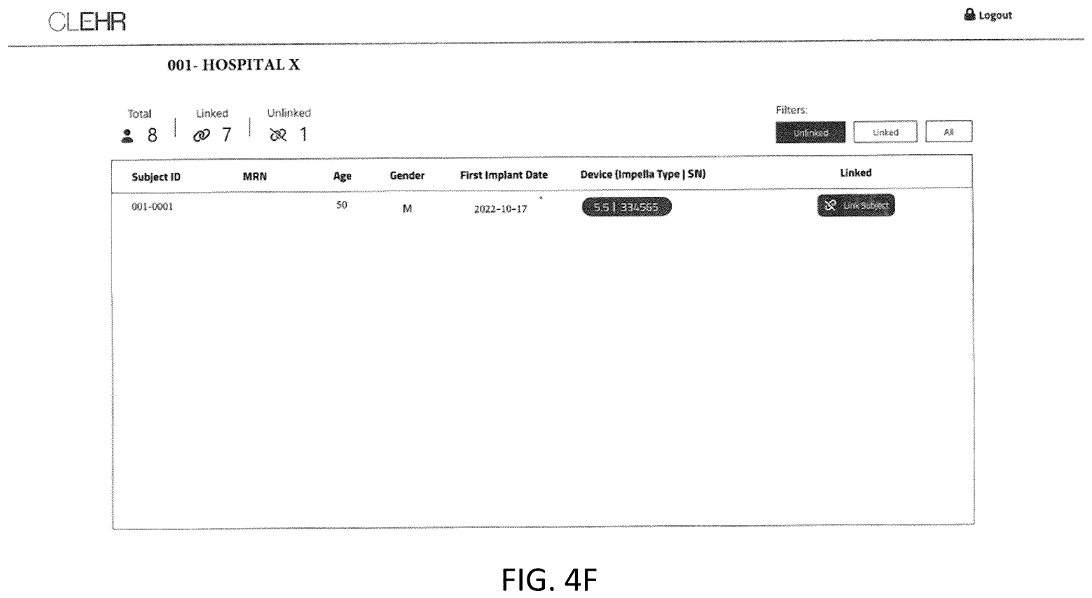

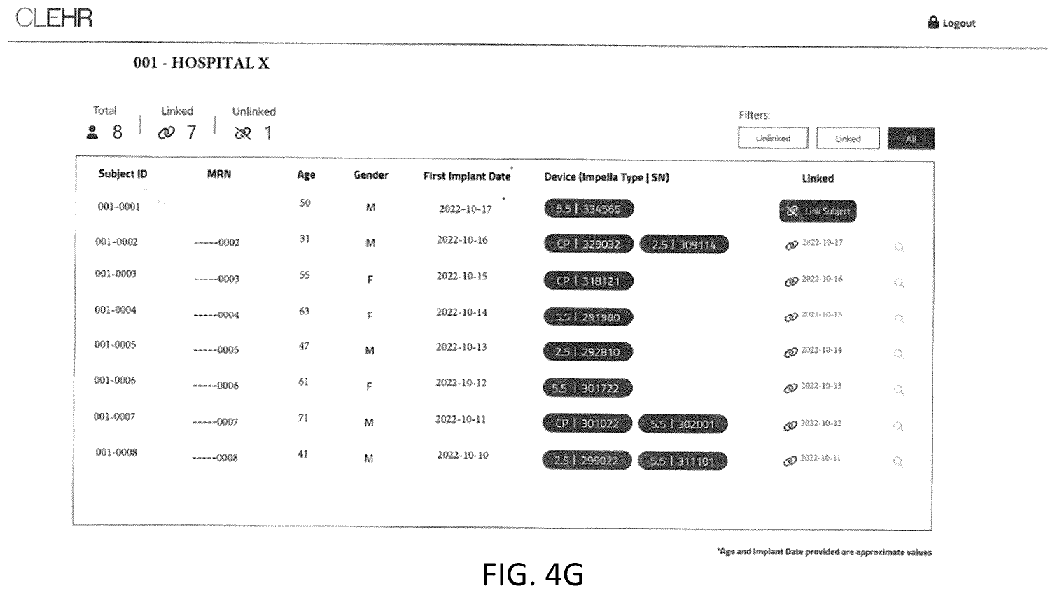

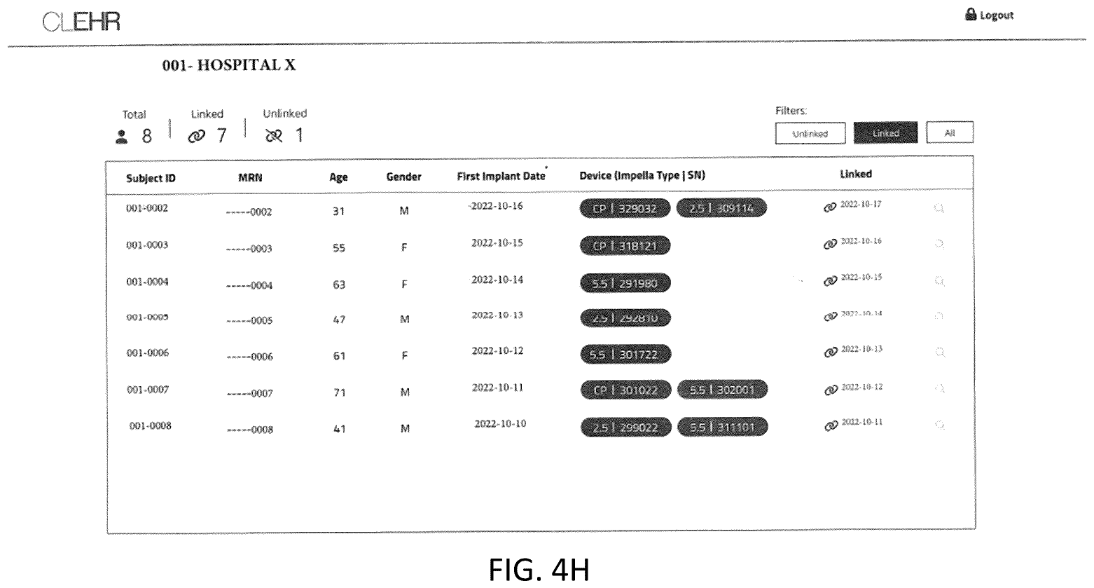

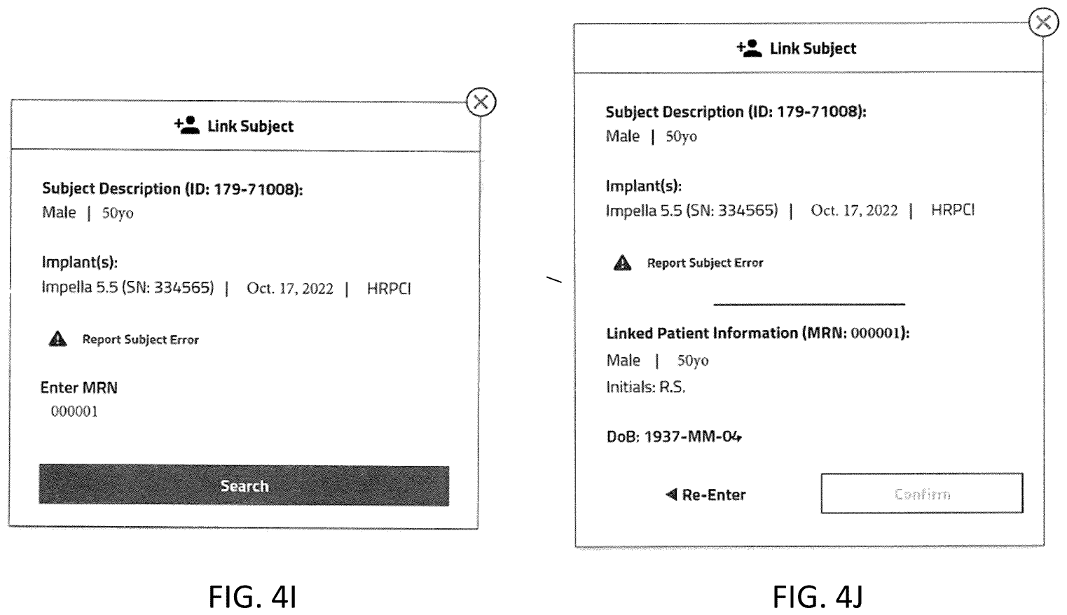

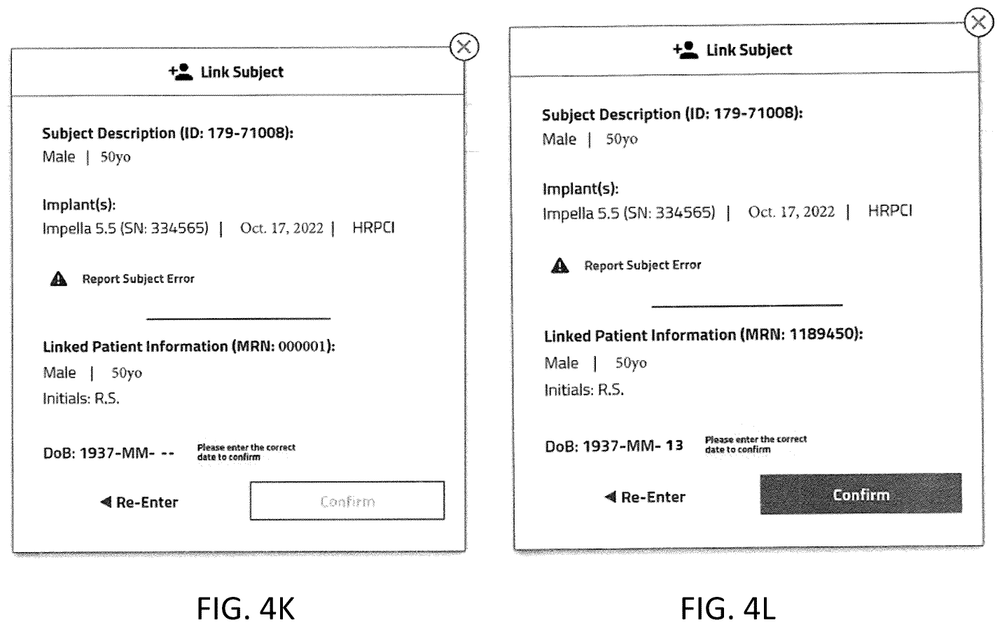

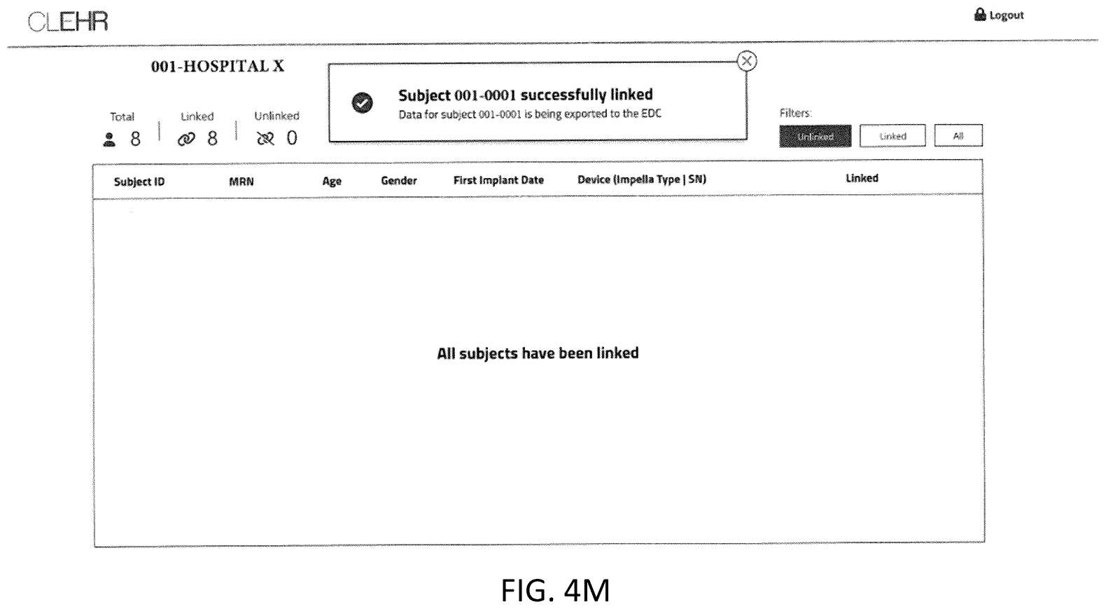

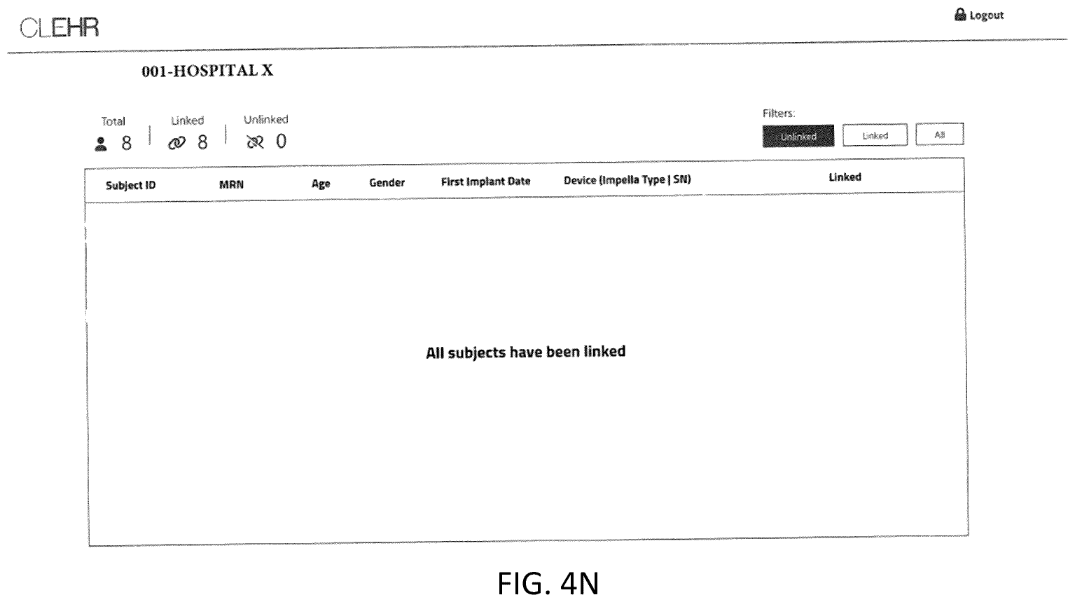

As shown in FIG. 1, system 100 may also include patient information extraction component 116 configured to determine when new subjects have been identified from subject ID source 112. For instance, patient information extraction component 116 may be configured to pull (e.g., periodically or in response to a request from a user) subject listing data from subject listing storage 114. In some embodiments, patient information extraction component 116 may be configured to present a graphical user interface (GUI) to a user. The GUI may include a representation of the subject listing data received from the subject listing storage 114. Portions of an example GUI that may be used in accordance with some embodiments are shown in FIGS. 4A-4N. A user interacting with the GUI may select one or more patients in the subject listing data to link a medical resource number (MRN) or some other patient identifier for the patient(s) (e.g., a Fast Healthcare Interoperability Resources (FHIR) identifier) to a patient identifier (PID) associated with an electronic data capture (EDC) system. An example process for linking a medical record patient identifier (e.g., an MRN) to a patient identifier in an electronic data capture (EDC) system is described with regard to the process shown in FIG. 3.

Upon linking of the MRN (or other patient identifier) to the PID for a patient, the patient information extraction component 116 may be configured to retrieve patient information (by using the MRN or other patient identifier) from an electronic medical record (EMR) for the patient. For instance, at least some of the information in the patient's EMR may be structured according to a particular healthcare data standard (e.g., HL7, FHIR), and patient information extraction component 116 may be configured to request data in accordance with the standard. As an example, patient information extraction component 116 may be configured to request particular FHIR data models that include information associated with a particular clinical trial. The EMR may include a standardized set of health data classes (e.g., Allergies and Intolerances, Medications, Vital Signs, etc.) and corresponding data elements (e.g., in accordance with the United States Core Data for Interoperability (USCDI) v1), and patient information extraction component 116 may be configured to request one or more of the data classes and/or data elements based on the standard.

In some embodiments, patient information extraction component 116 is configured to de-identify the retrieved patient information. For instance, the retrieved patient information may be de-identified according to the requirements of a particular clinical trial or based on some other criteria. In some embodiments, de-identification may include, but is not limited to, redacting information, shifting dates, masking fields, categorizing values, hashing values, or replacing values with a surrogate value. The de-identified patient information may then be stored, for example, in archive storage 118. For instance, patient information extraction component 116 may be configured to push de-identified patient information to archive storage 118. The de-identified patient information may also be provided to medical records storage 120. For instance, patient information extraction component 116 may be configured to push de-identified patient information to medical records storage 120. In some embodiments, transfer of de-identified patient information from patient information extraction component 116 to medical records storage 120 may be performed in accordance with one or more healthcare data standards, such as HL7, FHIR, etc.

In some embodiments, a window of data (e.g., corresponding to patient admission, device implant, procedure, researcher specific windows, etc.) to extract data from the patient's medical record may be determined (e.g., in response to a user request via a GUI, based on a clinical trial specification, etc.). In some embodiments, all information from the patient's medical record during the determined time window may be pulled from the patient's EMR. In other embodiments, only select information (e.g., particular FHIR data models or groups of FHIR data models) during the determined time window may be pulled from the patient's EMR.

System 100 may further include a data loader 122 configured to pull information from medical records storage 120. The pulled information may be provided to EDC system 124. In some embodiments, data loader 122 may be configured to transform information from medical records storage 120 to match a schema of a particular clinical trial associated with EDC system 124. For instance, data loader 122 may be configured to select only relevant pieces of the information to enter into the EDC system 124. Data loader 122 may then be configured to provide the transformed data to EDC system 124. In some embodiments, transformation of information from a patient's EMR may not be required. For example, if the information is stored in the patient's EMR according to particular standard (e.g. standardized FHIR data models), the information may be provided to the EDC from the EMR without transformation.

In some embodiments, an audit trail of all transactions may be maintained by system 100. At fixed intervals, at variable intervals based on workflow triggers, or on demand, the process of extracting information from the EMR, de-identifying it, storing it, and loading the information into the EDC system 124 can be repeated. The data can either be retrieved progressively (only updates) or cumulatively (all changes since start of a clinical trial window).

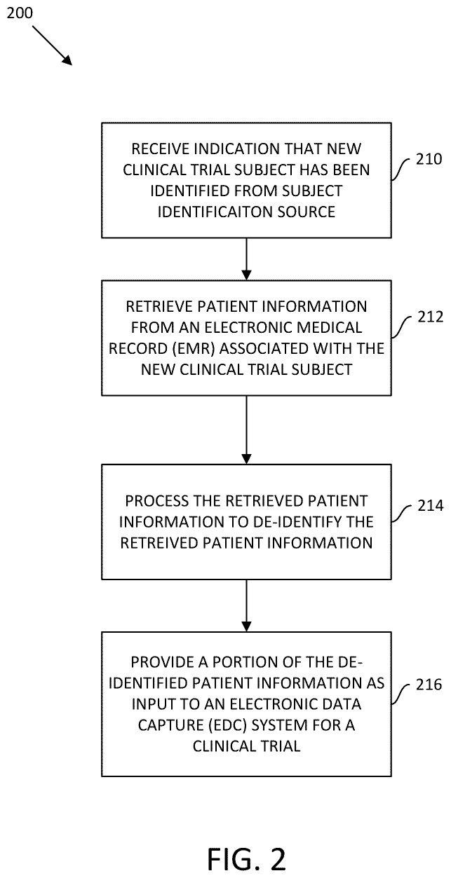

FIG. 2 is a flowchart of a process 200 for providing de-identified patient information from an EMR/EHR of a patient to an EDC, in accordance with some embodiments. Process 200 may begin in act 210, where an indication may be received that a new clinical trial subject has been identified from a subject identification source (e.g., subject ID source 112). Process 200 may then proceed to act 212, where patient information may be retrieved from an electronic medical record (EMR) associated with the new clinical trial subject. Between acts 210 and 212 in process 200, a link may be established between the subjects EMR and a record generated in an EDC. An example process for linking an EMR and an EDC record is described in connection with FIG. 3. Process 200 may then proceed to act 214, where the retrieved patient information may be de-identified for use in the clinical trial. Process 200 may then proceed to act 216, where a portion of the de-identified patient information may be provided as input to an EDC system for use in a clinical trial. For instance, when the subject's EMR and an EDC record are linked using one or more of the techniques described herein, information included in the subject's EMR may automatically be provided (periodically or in response to a user request) to the EDC system at any suitable interval during the duration of a clinical trial.

FIG. 3 is a flowchart of a process 300 for linking a patient's electronic medical record to a patient identifier in an electronic data capture system, in accordance with some embodiments. In act 310, a new patient at a clinical trial site may be identified. For instance, the clinical trial may relate to one or more outcomes associated with a treatment (e.g., patients having one or more implantable heart pump devices). When a patient at a clinical trial site is admitted for the treatment (e.g., implantation of a heart pump device), the admission may be captured by a patient identification system and/or a system associated with the implanted heart device (e.g., ImpellaConnect®). Process 300 may then proceed to act 312, where a case record and a patient identifier (PID) for the new patient may be generated in an electronic data capture (EDC) system associated with the clinical trial. Process 300 may then proceed to act 314, where a user at the clinical trial site (e.g., a clinical research coordinator, a healthcare provider) may be prompted to link the identified patient to the generated case record in the EDC system. For instance, the user may be instructed to access an electronic portal configured to display patients that have been identified but are not yet linked to patient identifiers in the EDC system. An example of an email communication sent to a user to link a new subject is shown in FIG. 4A. Prior to linking a subject, the user may be required to set up an online account for the electronic portal. An example of an email communication sent to a user to set up an online account is shown in FIG. 4B. The user may then be required to log-in to their account (e.g., using a username, password, temporary code, etc.). An example of an email communication sent to a user with a temporary code used to log-in to their account is shown in FIG. 4C. FIGS. 4D and 4E show portions of an example GUI for logging-in to an electronic portal, in accordance with some embodiments. After accessing the electronic portal, the user may be presented with information about the identified patient. FIG. 4F shows an example of a portion of a GUI displaying information about an identified clinical trial patient having an EMR that is not linked to an EDC record identifier. In the example of FIG. 4F, only information for a subset of patients (unlinked patients) is displayed on the GUI (e.g., the display is filtered to only show unlinked patients). FIG. 4G shows the portion of the GUI in FIG. 4F, in which all patients (both linked and unlinked patients) are shown on the GUI. As shown in FIG. 4G, all patients are associated with a subject ID in the electronic data capture (EDC) system, but only linked patients have a medical record patient identifier (e.g., MRN) specified. FIG. 4H shows the portion of the GUI in FIG. 4F, in which only information for linked patients is displayed on the GUI.

Continuing with process 300, after prompting the user to link a patient to a case record in act 314, process 300 may proceed to act 316, where the user may be requested to provide a medical record patient identifier (e.g., a medical resource number (MRN) or some other identifier for the patient, such as an FHIR ID) that can be used to link information in the patient's EMR to the generated patient identifier in the EDC system. FIG. 4I illustrates a portion of a GUI in which a user may be prompted to enter a MRN for the patient being linked. Upon receipt of the medical record patient identifier, process 300 may proceed to act 318, where information about the patient may be retrieved from a connected EMR system using the medical record patient identifier. Process 300 may then proceed to act 320, where the retrieved information may be presented to the user for confirmation of its accuracy. FIG. 4J shows a portion of a GUI in which the user is asked to confirm the accuracy of information retrieved from a patient's EMR. As shown in FIG. 4J, the user may also be prompted to correct information that the system determines may be incorrect. FIGS. 4K and 4L show additional examples of portions of a GUI for confirming/correcting retrieved information from a patient's EMR, in accordance with some embodiments. Upon confirmation of the accuracy of the retrieved information, process 300 may proceed to act 322, where a link between the medical record patient identifier and the patient identifier in the EDC may be established. FIG. 4M shows a portion of a GUI, which displays that the EMR for a patient has been successfully linked to a record in the EDC system. After establishment of the link, de-identified data from the patient's medical record may be automatically transferred into the EDC system, as described herein. FIG. 4N shows a portion of the GUI, which displays that there are no remaining unlinked patients to link.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of extracting patient information from an electronic medical record (EMR) for use in a clinical trial, the method comprising:
receiving an indication that a new clinical trial subject has been identified from a subject identification source;
retrieving, from an EMR associated with the new clinical trial subject, patient information;
processing the retrieved patient information to de-identify the retrieved patient information thereby generating de-identified patient information; and
providing a portion of the de-identified patient information as input to an electronic data capture (EDC) system for the clinical trial.

2. The method of claim 1, further comprising:
issuing a request to a subject listing storage component for current subject listing data,
wherein the indication that a new clinical trial subject has been identified is received in response to issuing the request.

3. The method of claim 2, wherein the subject listing storage component is configured to pull patient information from the subject identification source to generate the current subject listing data.

4. The method of claim 1, wherein the subject identification source comprises a data source configured to store a plurality of subjects having an implantable medical device.

5. The method of claim 4, wherein the implantable medical device is an implantable heart pump.

6. The method of claim 1, further comprising:
displaying a graphical user interface (GUI) on a computing device, wherein the graphical user interface includes a representation of the new clinical trial subject.

7. The method of claim 6, further comprising:
receiving via the GUI, a request to link a medical record patient identifier for the new clinical trial subject to a patient identifier in the EDC system; and
linking the medical record patient identifier to the patient identifier in the EDC system in response to receiving the request.

8. The method of claim 7, wherein the request includes the medical record patient identifier, and linking the medical record patient identifier to the patient identifier in the EDC system comprises:
retrieving from an EMR system, patient information associated with the medical record patient identifier;
displaying the retrieved patient information on the GUI;
receiving, via the GUI, confirmation that the retrieved patient information is accurate; and
linking the medical record patient identifier with the patient identifier in the EDC system in response to receiving confirmation that the retrieved patient information is accurate.

9. The method of claim 8, wherein linking the medical record patient identifier to the patient identifier in the EDC system further comprises:
generating for the new clinical trial subject, a case record and the patient identifier in the EDC system.

10. (canceled)

11. The method of claim 1, further comprising:
storing the de-identified patient information in a data store,
wherein providing a portion of the de-identified patient information as input to an EDC system for the clinical trial comprises providing a portion of the de-identified patient information from the data store.

12. The method of claim 11, wherein storing the de-identified patient information in a data store comprises transferring the de-identified patient information to the data store in accordance with one or more healthcare data transfer standards.

13. The method of claim 12, wherein the one or more healthcare data transfer standards include HL7 or FHIR.

14. The method of claim 1, wherein providing a portion of the de-identified patient information as input to an EDC system comprises providing less than all of the de-identified patient information stored in the data store as input to the EDC system.

15. The method of claim 1, wherein processing the retrieved patient information to de-identify the retrieved patient information comprises de-identifying the retrieved patient information based, at least in part, on requirements of the clinical trial.

16. The method of claim 1, wherein processing the retrieved patient information to de-identify the retrieved patient information comprises one or more of redacting information, shifting dates, masking data fields, categorizing data values, hashing data values, or replacing data values with surrogate values.

17. The method of claim 1, wherein retrieving patient information from an EMR associated with the new clinical trial subject comprises retrieving patient information within a particular time window.

18. The method of claim 17, wherein retrieving patient information within a particular time window comprises retrieving all patient information from the EMR within the particular time window.

19. The method of claim 17, wherein the particular time window is selected from the group consisting of a medical facility admission window, a medical device implant procedure window, and a researcher-specified window.

20. The method of claim 1, further comprising:
maintaining an audit trail of all transactions associated with retrieving the patient information, generating the de-identified patient information and providing the portion of the de-identified patient information as input to the EDC system.

21. A computerized system for automatically transferring data from an electronic medical record (EMR) system to an electronic data capture (EDC) system for use in a clinical trial, the computerized system comprising:
a subject identification source configured to store an identifier for each of one or more potential clinical trial subjects;
subject listing storage configured to pull patient information from the subject identification source to generate a current subject listing data; and
a hardware processor programmed to:
request, from the subject listing storage, the current subject listing data;
retrieve, from an EMR associated with one or more clinical trial subjects included in the current subject listing data, patient information, wherein the EMR is included in the EMR system; and
process the retrieved patient information to de-identify the retrieved patient information thereby generating de-identified patient information; and
provide a portion of the de-identified patient information as input to the EDC system for the clinical trial.

22-38. (canceled)
